(19) United States
(12) Patent Application Publication
(10) Pub. No.: US 2024/0075059 A1
(43) Pub. Date: Mar. 7, 2024

(54) METHODS OF SELECTIVELY PROMOTING GASTROINTESTINAL MICROBIAL GROWTH

(71) Applicant: DSM IP Assets B.V., Heerlen (NL)

(72) Inventors: Joshua Thomas CLAYPOOL, Kaiseraugst (CH); Ghislain SCHYNS, Kaiseraugst (CH); Carrie Louise WALK, Kaiseraugst (CH)

(21) Appl. No.: 18/261,762

(22) PCT Filed: Jan. 19, 2022
(86) PCT No.: PCT/EP2022/051106

§ 371 Date: Jul. 17, 2023

Related U.S. Application Data
(60) Provisional application No. 63/138,942, filed on Jan. 19, 2021.

(51) Int. Cl. A61K 33/04; A23K 20/105; A23K 20/174; A23K 20/20; A23K 50/75; A61K 9/00; A61K 31/355; A61P 31/04

(52) U.S. Cl. CPC A61K 33/04; A23K 20/105; A23K 20/174; A23K 20/30; A23K 50/75; A61K 9/0056; A61K 31/355; A61P 31/04

(57) ABSTRACT

The present disclosure relates to methods of feeding animals by providing feed additives that modulate the gut microbiome to improve the health, nutrition, and growth performance. The present disclosure further relates to methods of modulating metabolites present in the gastrointestinal tract of an animal. Such modulation includes, for example, modulating the level said metabolites.

### BACKGROUND

The gut microbiome, e.g., bacteria, viruses, fungi, mold, protozoa, etc. that reside in the digestive track, is responsible for converting undigested and unabsorbed components of an animal's diet into thousands of biologically active metabolites. These metabolites interface in turn with the local and systemic physiology of the animal as well as the animal's external environment.

Under normal circumstances, the biochemical output of the microbiome is dictated in part by the composition of food consumed by the animal and in part by the phylogenetic composition of the gut microbiome. In a conventional diet, particularly one comprising plant-fiber polysaccharides and species such as cellulose, lignin, hemicellulose, pectins, and starch-bound protein, a portion of the food consumed by the animal remains undigested and unabsorbed by the primary digestive process. These unabsorbed species reach the lower intestinal system, where they can be processed and utilized by the microbiota and converted to metabolites. The resulting gut metabolome generated by the metabolic action of the gut microbiome on these unabsorbed components of the feed is affected by the chemical compositions of those various unabsorbed components.

Metabolites produced in the gut can be absorbed, for example through the colonic or portal circulatory systems, and transported to other organs of the animal where they can affect the structure and/or function of those organs. These biochemicals in turn affect diverse biological functions, such as nutrient absorption, energy regulation, mitochondrial function, systemic inflammation, stress response, liver function, kidney function, cardiometabolic function, satiety, mood, and alertness. Metabolites produced in the gut can also be excreted by the animal to its external environment.

In some cases, the metabolites produced by the gut microbiome are beneficial to the host or otherwise contribute to the productivity, health, welfare and sustainability of the host animal. In other cases, the metabolites produced by the gut microbiome are detrimental to the host and result in decreased productivity, health, or welfare. Certain metabolites are undesirable because they are detrimental to the external environment of the animal when excreted, and can result in water, soil, and/or atmospheric pollution, or otherwise increase the environmental footprint of raising the animals.

It is well know that prebiotics like oligosaccharides or probiotics like Lactobacillus and Bacilli are able to impact/influence the composition of the broiler gut microbiome in such a way that beneficial microorganisms are more abundant or metabolic pathways of interest, like the ones involved into the biosynthesis of short chain fatty acids such as butyrate or acetate for example are more active.

It is also well known that some of the above mentioned eubiotic products are also able to reduce the abundance of non-desirable enterobacteria such as Escherichia coli or Salmonella.

There is thus still a need to be able to modulate or otherwise control the metabolic pathways and metabolic output of the gut microbiome in animals for the purpose of improving nutrition, health, welfare and/or sustainability of production animals and companion animals. The challenge, however, is that animals typically exhibit high taxonomic variability in the phylogenetic composition of their gut microbiomes.

Surprisingly, the inventors of the present invention found that combinations of vitamin E and selenium provide beneficial effects when used in an animal feed or an animal feed additive. In particular feed supplied with such a combination can modify/modulate the microbiome of the animal and metabolic pathways associated therewith as for example pathways to produce short chain fatty acids. The supplied combination can therefore improve performance of animal production via influencing the composition of the animal gut microbiome and/or modulation of pathways present in the microbiome.

Vitamin E, as a dietary essential fat-soluble vitamin, can improve animal performance when provided in amounts above minimal requirements. Due to the potent antioxidant properties of tocopherols, the impact of α-tocopherol in the prevention of chronic diseases believed to be associated with oxidative stress has often been studied and beneficial effects have been demonstrated (Brigelius-Flohe, R. and Traber, M. G., The FASEB Journal, 1999, 13(10): 1145-1155.). The biological effects of vitamin E are mostly seen in the prevention of resorption of fetuses, testicular degeneration, muscle dystrophy, anemia and encephalomalacia; the classical signs of vitamin E deficiency in animals. The influence of vitamin E on the immune system has also become an important issue (Politis, I. et al., Amer. J. Veter. Res., 1995, 56(2): 179-184.). It was recommended that, vitamin E at 6 to 20 times the NRC-recommended concentrations would improve the immune response of farm animals (Nockels, C. F., Recent Advances in Animal Nutr., 177., 1986).

Selenium (Se) is an important trace mineral for farm animals, which prevents the oxidation of membrane polyunsaturated fatty acids and DNA by oxygen radicals produced throughout aerobic metabolism (Koyuncu, M. and Yerlikaya, H., South Afr. J. Animal Sci., 2007, 37(4): 233-236.). Se has a biological function related to vitamin E in that it is an important component of glutathion peroxidase, an enzyme involved in detoxification of hydrogen peroxide and lipid hydroperoxides.

Furthermore, Se is a component of selenoproteins and is involved in immune function in farm animals (Meschy, F., Livestock Production Science, 2000, 64(1): 9-14.). Se deficiency plays a role in several economically important livestock diseases; problems that include decreased fertility, abortion, retained placenta and neonatal weakness (McDowell, L. R. et al., Animal Feed Sci. Technol., 1996, 60(3): 273-296.).

It was reported that a high level of Se and vitamin E (VitE) in heifers in late pregnancy had positive effect on immune system (Moeini, M. M. et. Al., Biological Trace Element Res., 2011, 144(1-3): 529-537.). It has also been reported that, the mean milk yield and milk composition of cows receiving supplemental inorganic selenium (as sodium selenite) and organic selenium (as selenium yeast) were greater for cows receiving organic selenium (Silvestre et al. 2007, Effect of selenium source on production, reproduction, and immunity of lactating dairy cows. In: Florida Ruminant Nutrition Symposium. Gainesville, FL.).

### SUMMARY OF THE INVENTION

Provided herein are methods of promoting the growth of one or more beneficial microbial genuses in the gastrointestinal tract of an animal, comprising: administering a nutritional composition (animal feed) comprising a base nutritional composition and a Se-VitE preparation described herein to an animal, wherein the level of one or more microbial genuses in the gastrointestinal tract of the animal is higher relative to the level of the microbial genuses in the gastrointestinal tract of an animal administered a nutritional composition lacking the Se-VitE preparation or relative to the level of the microbial genuses in the gastrointestinal tract of the animal prior to administration of the nutritional composition comprising the Se-VitE preparation.

Provided herein are also methods of suppressing the growth of one or more non-beneficial microbial genuses in the gastrointestinal tract of an animal, comprising: administering a nutritional composition comprising a base nutritional composition and a Se-VitE preparation described herein to an animal, wherein the level of one or more microbial genuses in the gastrointestinal tract of the animal is lower relative to the level of the microbial genuses in the gastrointestinal tract of an animal administered a nutritional composition lacking the Se-VitE preparation or relative to the level of the microbial genuses in the gastrointestinal tract of the animal prior to administration of the nutritional composition comprising the Se-VitE preparation.

In another aspect, provided herein are methods of improving the nutrition, health, welfare, and sustainability of animals by providing to the animals feed additives that increase or decrease the expression of one or more metabolic pathways in the metagenome of the animal's microbiome. In certain embodiments, the methods of improving the nutrition of the animal comprise increasing the abundance of, expression of, or flux through metabolic pathways in the metagenome of the gastrointestinal microflora that are responsible for harvesting nutritional energy from undigested components of the animal's diet.

In one embodiment, the invention is related to a method of improving nitrogen utilization in an animal, the method comprising: administering a nutritional composition comprising a base nutritional composition and a Se-VitE preparation to said animal, wherein the level of a plurality of metabolites associated with enhanced nitrogen utilization is higher in a gastrointestinal sample from said animal compared to a gastrointestinal sample from a comparable control animal that has been administered a comparable nutritional composition comprising said base nutritional composition and lacking said Se-VitE preparation.

In a second embodiment, the invention is related to a method of improving carbon utilization in an animal, the method comprising: administering a nutritional composition comprising a base nutritional composition and a Se-VitE preparation to said animal, wherein the level of a plurality of metabolites associated with improved carbon utilization in a gastrointestinal sample from said animal is higher compared to a comparable control animal that has been administered a comparable nutritional composition comprising said base nutritional composition and lacking said Se-VitE preparation.

In a preferred embodiment of the invention, said plurality comprises at least 2, 3, 4, 5, 6, 7, 8, 9, or 10 metabolites selected from the group consisting of (R)-lactate, (R)-lactoyl-CoA, (S)-lactate, (S)-propane-1,2,-diol, 1-propanal, acetate, acetyl-CoA, acryloyl-CoA, propanoate, propanoyl-CoA, and pyruvate.

Provided herein are also methods of improving animal health, comprising: administering a nutritional composition comprising a base nutritional composition and a Se-VitE preparation described herein to the animal, and wherein the administering results in at least one of a) improving the gastrointestinal microbiota of the animal, b) selectively increasing the relative abundance of beneficial bacteria in the gastrointestinal microbiota of the animal, c) selectively decreasing the relative abundance of non-beneficial bacteria in the gastrointestinal microbiota of the animal.

In some embodiments, the nutritional composition comprising the Se-VitE preparation is administered to the animal for at least 1, 7, 10, 14, 30, 45, 60, 90, or 120 days. In some embodiments, the nutritional composition comprising the Se-VitE preparation is administered to the animal at least once, twice, three, four, or five times a day. In some embodiments, administering comprises providing the nutritional composition to the animal to ingest at will, wherein the animal ingests at least a portion of the nutritional composition in every 24 hours period.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWING

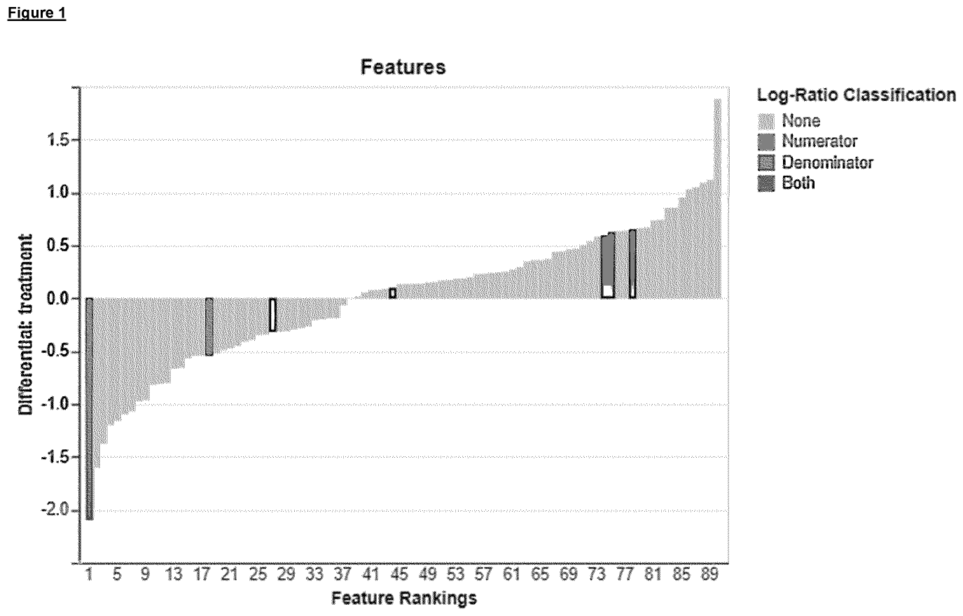

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawing (FIG. 1), which shows a rank plot with features positively or negatively associated to treatment with vitE and Se.

### Definitions

As used herein the term “administering” includes providing a Se-VitE preparation, a nutritional composition (animal feed composition) described herein, to an animal such that the animal may ingest the Se-VitE preparation, the nutritional composition, or the animal feed composition at will. In some embodiments, the animal ingests some portion of the Se-VitE preparation, the nutritional composition, or the animal feed composition in every 24 hour period or every other 24 hour period for at least 7 days, 14 days, 21 days, 30 days, 45 days, 60 days, 75 days, 90 days or 120 days.

As used herein, the term “Se-VitE preparation” may refer to a blend or feed premix comprising Selenium, Vitamin E or a Vitamin E derivative as for example Vitamin E acetate, and optionally other feed additives.

The nutritional composition according to the present invention is an animal feed composition as described below.

The base nutritional composition according to the present invention is an animal feed additive or animal feed premix as described below.

In the present invention, the term “animal feed additive” refers to an ingredient or combination of ingredients added to the animal feed, usually used in micro quantities and requires careful handling and mixing. Such ingredient includes but is not limited to vitamins, amino acids, minerals, enzymes, eubiotics, colouring agents, growth improving additives and aroma compounds/flavourings, polyunsaturated fatty acids (PUFAs); reactive oxygen generating species, antioxidants, anti-microbial peptides, anti-fungal polypeptides and mycotoxin management compounds etc.

Animal: The term “animal” refers to any animal except humans. Examples of animals are monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); pets such as cats and dogs; horses (including but not limited to hotbloods, coldbloods and warm bloods) crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: In the present invention, the term “animal feed” or “feed” refers to any compound, preparation, or mixture suitable for or intended for intake by an animal and capable of maintaining life and/or promoting production of the animal without any additional substance being consumed except water. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Feed Premix: The incorporation of the composition of feed additives as exemplified herein above to animal feeds, for example poultry feeds, is in practice carried out using a concentrate or a premix. A premix designates a preferably uniform mixture of one or more microingredients with diluent and/or carrier. Premixes are used to facilitate uniform dispersion of micro-ingredients in a larger mix. A premix according to the invention can be added to feed ingredients or to the drinking water as solids (for example as water soluble powder) or liquids.

Nutrient: The term “nutrient” in the present invention means components or elements contained in dietary feed for an animal, including water-soluble ingredients, fat-soluble ingredients and others. The example of water-soluble ingredients includes but is not limited to carbohydrates such as saccharides including glucose, fructose, galactose and starch; minerals such as calcium, magnesium, zinc, phosphorus, potassium, sodium and sulfur; nitrogen source such as amino acids and proteins, vitamins such as vitamin B1, vitamin B2, vitamin B3, vitamin B6, folic acid, vitamin B12, biotin and phatothenic acid. The example of the fat-soluble ingredients includes but is not limited to fats such as fat acids including saturated fatty acids (SFA); mono-unsaturated fatty acids (MUFA) and poly-unsaturated fatty acids (PUFA), fibre, vitamins such as vitamin A, vitamin E and vitamin K.

### DETAILED DESCRIPTION OF THE INVENTION

As mentioned above, the inventors of the present invention surprisingly found that combinations of vitamin E and selenium provide beneficial effects when used in an animal feed or an animal feed additive. In particular feed supplied with such a combination can modify/modulate the microbiome of the animal and metabolic pathways associated therewith as for example pathways to produce short chain fatty acids. The supplied combination can therefore improve performance of animal production via influencing the composition of the animal gut microbiome and/or modulation of pathways present in the microbiome.

Vitamin E may be any one or more selected from the group consisting of d-α-tocopherol, dl-α-tocopherol, d-α-tocopherol acetate, dl-α-tocopherol acetate, d-α-tocopherol succinate and/or dl-α-tocopherol succinate. Vitamin E may be synthesized or obtained from commercial sources. Example of commercially available vitamin E is ROVIMIX® E50.

Selenium may be in an inorganic form such as sodium selenite (Na₂SeO₃) and sodium selenite (Na₂SeO₄), or in an organic form such as selenomethionine. Selenium may be obtained from any source, and a composition thereof may be prepared using convenient technology selenium. Examples of commercially available selenium products are MICROGRAN® Se, SELSAF® 2000 and Biomin® TorSel™ 4000.

In the present invention, the Se-VitE preparation may be formulated as a liquid formulation or a solid formulation. Accordingly, the composition according to the present invention may also comprise one or more formulating agents.

The formulating agents may be selected from the group consisting of polyol such as glycerol, sorbitol, ethylene glycol, diethylene glycol, triethylene glycol, 1,2-propylene glycol, 1,3-propylene glycol, dipropylene glycol and polyethylene glycol (PEG); a salt such as organic or inorganic zinc, sodium, potassium, calcium or magnesium salts (for example, magnesium sulfate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate and zinc sulfate); and starch or a sugar or sugar derivative such as sucrose, dextrin, glucose, lactose and sorbitol; small organic molecules, flour, cellulose and minerals and clay minerals (also known as hydrous aluminium phyllosilicates such as kaolinite or kaolin).

The Se-VitE preparation according to the present invention may also comprise one or more emulsifying agents. The emulsifying agents may be selected advantageously from the group consisting of polyglycerol esters of fatty acids such as esterified ricinoleic acid or propylene glycol esters of fatty acids, saccharo-esters or saccharo-glycerides, polyethylene glycol, lecithins, etc.

In the preparation according to the present invention, vitamin E may be provided in an amount of from 10 mg to 500 mg, preferably from 20 mg to 450 mg, more preferably from 25 mg to 400 mg such as 25, 30, 35, 40, 45, 50, 60, 70, 80, 90, 100, 120, 140, 160, 180, 200, 250, 300, 350 and 400 mg, per 1 Kg of the final animal feed.

In the preparation according to the present invention, selenium may be provided in an amount of from 0.1 mg to 5 mg, preferably from 0.2 mg to 4.0 mg, more preferably from 0.3 mg to 3.0 mg, more preferably from 0.5 mg to 2.5 mg, even more preferably from 0.8 mg to 2.0 mg such as 0.8, 0.85, 0.9, 0.95, 1.0, 1.05, 1.1, 1.15, 1.2, 1.25, 1.3, 1.35, 1.4, 1.45, 1.5, 1.6, 1.7, 1.8, 1.9 and 2.0 mg, per 1 Kg of the final animal feed.

As anticipated by any person skilled in the art, the Se-VitE preparation according to the present invention may be formulated as an animal feed premix. Accordingly, the present invention also provides an animal feed premix which comprises the preparation comprising vitamin E and selenium according to the present invention. The preparation or the animal feed premix according to the the present invention may further includes micro-ingredients.

The micro-ingredients include but are not limited to aroma compounds; antimicrobial peptides; polyunsaturated fatty acids (PUFAs); reactive oxygen generating species; at least one enzyme, and fat- and water-soluble vitamins, as well as minerals.

Examples of antimicrobial peptides (AMP's) are CAP18, Leucocin A, Protegrin-1, Thanatin, Defensin, Lactoferrin, Lactoferricin, and Ovispirin such as Novispirin (Robert Lehrer, 2000), Plectasins, and Statins.

Examples of polyunsaturated fatty acids are C₁₈-, C₂₀- and C₂₂-polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Examples of enzyme are phytase (EC 3.1.3.8 or 3.1.3.26); xylanase (EC 3.2.1.8); galactanase (EC 3.2.1.89); alpha-galactosidase (EC 3.2.1.22); protease (EC 3.4., phospholipase A 1 (EC 3.1.1.32); phospholipase A2 (EC 3.1.1.4); lysophospholipase (EC 3.1.1.5); phospholipase C (EC 3.1.4.3); phospholipase D (EC 3.1.4.4); amylase such as, for example, alpha-amylase (EC 3.2.1.1); and/or beta-glucanase (EC 3.2.1.4 or EC 3.2.1.6).

Examples of fat-soluble vitamins include but are not limited to vitamin A, vitamin D₃, and vitamin K, e.g. vitamin K₃.

Examples of water-soluble vitamins include but are not limited to vitamin B₁₂, biotin and choline, vitamin B₁, vitamin B₂, vitamin B₆, niacin, folic acid and panthothenate, e.g. Ca-D-panthothenate.

Examples of minerals include but are not limited to calcium, phosphorus, sodium, potassium, magnesium, chlorine, iodine, iron, manganese, copper, molybdenum, cobalt and zinc. Common mineral supplements in feed are: Limestone, Bone meal, Oyster shell, Sodium chloride, Dicalcium phosphate, Manganese sulphate, Potassium iodide, and Superphosphate. Sources of minerals include meat scraps, fish meal, milk products, ground limestone (calcium), ground oyster shells (calcium), dicalcium phosphate (calcium, phosphorus), defluorinated rock phosphate (phosphorus, calcium), steamed bone meal (phosphorus, calcium), salt (sodium, chlorine, iodine), manganese sulfate (manganese), manganese oxide (manganese), zinc carbonate (zinc), zinc oxide (zinc).

Any person skilled in the art should understand that, the Se-VitE preparation or the animal feed premix according to the the present invention may be incorporated into an animal feed. Accordingly, the present invention also provides an animal feed which comprises the Se-VitE preparation as described above.

As anticipated by any person skilled in the art, the animal feed according to the the present invention may further include any number of components typical for an animal feed, such as proteins, carbohydrates, fats and additional additives.

In the animal feed according to the present invention, vitamin E may be contained in an amount of from 10 mg to 500 mg, preferably from 20 mg to 450 mg, more preferably from 25 mg to 400 mg such as 25, 30, 35, 40, 45, 50, 60, 70, 80, 90, 100, 120, 140, 160, 180, 200, 250, 300, 350 and 400 mg, per 1 Kg of the animal feed.

In the animal feed according to the present invention, selenium may be contained in an amount of from 0.1 mg to 5 mg, preferably from 0.2 mg to 4.0 mg, more preferably from 0.3 mg to 3.0 mg, more preferably from 0.5 mg to 2.5 mg, even more preferably from 0.8 mg to 2.0 mg such as 0.8, 0.85, 0.9, 0.95, 1.0, 1.05, 1.1, 1.15, 1.2, 1.25, 1.3, 1.35, 1.4, 1.45, 1.5, 1.6, 1.7, 1.8, 1.9 and 2.0 mg, per 1 Kg of the animal feed.

In a first aspect, the methods described herein include increasing the gut relative abundance of beneficial gut microorganisms or selectively enhancing or promoting the growth of one or more beneficial microbial genuses in the gastrointestinal tract of an animal. In some embodiments, the make-up of the gastrointestinal microbiota of an animal is shifted by the Se-VitE preparation toward that of a healthy state.

In some embodiments, the beneficial microbial genus is a bacteria, bacteriophage, fungi or protozoan genus.

In a preferred embodiment, the level of 1, 2, 3, 4, 5 or more microbial genuses in the gastrointestinal tract of the animal is higher relative to the level of the microbial genuses in the gastrointestinal tract of an animal administered a nutritional composition lacking the Se-VitE preparation.

In accordance with the present invention genuses are groups of species. Preferred genuses are Lactobacillus and Faecalibacterium, wherein Lactobacillus genus contains for example Lactobacillus reuteri and Lactobacillus rhamnosus and Faecalibacterium genus contains Faecalibacterium prausnitzi.

In some specific embodiments, the microbial genuses belongs to the phylum “Firmicutes”. Preferred “Firmictus” genuses are Lactobacilli, Coprococcus, Faecalibacterium.

Firmicutes of interest are producers of primary metabolites such as short chain fatty acids, lactate etc. Firmicutes of interest are also producers of secondary metabolites such as bacteriocins, microbiome neuromodulators or of extracellular enzymes such carbohydrate-linked enzymes, bile acid degrading enzymes etc.

Increased or decreased levels of primary and secondary metabolites are very often associated with animal performance, health and welfare, for example with an enhanced or suppressed carbon- or nitrogen utilization.

Therefore, in a second aspect, the methods described herein include selectively promoting or inhibiting the production of one or more gastrointestinal metabolites in an animal.

In some embodiments, the one or more of the metabolites is a primary metabolite. Primary metabolites include but are not limited to metabolites associated with the C3 microbiome pathway e.g., (R)-lactate, (R)-lactoyl-CoA, (S)-lactate, (S)-propane-1,2,-diol, 1-propanal, acetate, acetyl-CoA, acryloyl-CoA, propanoate, propanoyl-CoA, and pyruvate; metabolites associated with the Energy Metabolism microbiome pathway e.g., 2-oxoglutarate, fumarate, L-alanine, L-glutamate, oxaloacetate, propanoyl-CoA, pyruvate, and succinate; metabolites associated with the Adverse Amino Acid Degradation microbiome pathway e.g., (3S,5S)-3,5-diaminohexanoate, (S)-3-methyl-2-oxopentanoate, (S)-5-amino-3-oxohexanoate, 2-oxoglutarate, acetyl-CoA, ammonia, D-alanine, Formate, Fumarate, Glycine, L-2-amino-3-oxobutanoate, L-alanine, L-asparagine, L-aspartate, L-glutamate, L-isoleucine, N-formimino-L-glutamate, N-formyl-L-glutamate, N2-succinylglutamate, and Pyruvate; and metabolites associated with the C4 Pathway microbiome pathway e.g., (3R)-3-hydroxybutanoyl-CoA, (R)-lactate, (R)-lactoyl-CoA, (S)-3-aminobutanoyl-CoA, (S)-3-hydroxy-isobutanoate, (S)-3-hydroxy-isobutanoyl-CoA, (S)-3-hydroxybutanoyl-CoA, (S)-5-amino-3-oxohexanoate, (S)-lactate, 4-hydroxybutanoate, Acetate, Acetoacetate, acetoacetyl-CoA, acetyl-CoA, butanoate, butanoyl-CoA, coenzyme_A, crotonyl-CoA, succinate, succincate_semialdehyde, and succinyl-CoA.

In some embodiments, the one or more of the metabolites is a secondary metabolite. Secundary metabolites include but are not limited to bacteriocins, microbiome neuromodulators.

In a third aspect, the methods described herein include promoting expression of one or more microbial (e.g., bacterial) protein in the gastrointestinal tract of an animal. In some embodiments, the microbial protein is a bacterial protein. In certain embodiments the microbial (e.g., bacterial) protein is a hydrolytic enzyme, a protein involved in digestion (e.g. hydrolytic enzymatic digestion), or a protein involved in metabolism. Microbial proteins include, but are not limited to carbohydrate active enzymes (CAZymes) and bile acid degrading enzymes.

In a further aspect, the methods described herein include decreasing the gut relative abundance of non-beneficial gut microorganisms and pathogens or selectively suppressing or decreasing the growth of one or more non-beneficial microbial genuses in the gastrointestinal tract of an animal. Non-beneficial microorganisms according to the present invention are Proteobacteria as for example Escherichia coli and Bacteroidetes as for example Bacteroides and Prevotella.

In a preferred embodiment, the level of 1, 2, 3, 4, 5 or more non-beneficial microbial genuses in the gastrointestinal tract of the animal is lower relative to the level of the microbial genuses in the gastrointestinal tract of an animal administered a nutritional composition lacking the Se-VitE preparation.

In certain embodiments, the methods described herein comprise delivering or increasing one or more gastrointestinal metabolites in a gastrointestinal tract of an animal. In some embodiments, the metabolites comprise short chain fatty acids (SCFAs), nitrogenous metabolites, metabolites of the carbon pathways, for example amino acids, pyruvic acid, butyric acid, propionic acid, acetic acid, lactic acid, valeric acid, isovaleric acid any combination thereof.

In some embodiments, a level of one or more metabolites in the gastrointestinal tract of the animal that is administered the nutritional composition comprising the Se-VitE preparation is higher relative to a level of the metabolite in the gastrointestinal tract of an animal administered a nutritional composition lacking the Se-VitE preparation.

For example, in some specific embodiments, the level of butyric acid in the gastrointestinal tract of the animal that is administered the nutritional composition comprising the Se-VitE preparation is higher relative to a level of butyric acid in the gastrointestinal tract of an animal administered a nutritional composition lacking the Se-VitE preparation. In some specific embodiments, the level of propionic acid in the gastrointestinal tract of the animal that is administered the nutritional composition comprising the Se-VitE preparation is higher relative to a level of propionic acid in the gastrointestinal tract of an animal administered a nutritional composition lacking the Se-VitE preparation.

In some embodiments, a level of 2, 3, 4, 5, 6, 7, 8, 9, 10, or more metabolites in the gastrointestinal tract of the animal that is administered the nutritional composition comprising the Se-VitE preparation are each higher relative to a level of the metabolite in the gastrointestinal tract of an animal administered a nutritional composition lacking the Se-VitE preparation.

In certain embodiments, the methods described herein pertain to increasing the expression of microbiome metagenomic functions that translate to a nutritional, health, or welfare benefit in the host animal. In some embodiments, the microbiome metagenomic functions comprise one or more metabolic pathways or groups of pathways (e.g., superpathways). In certain embodiments, the microbiome metagenomic function comprises pathways that produce metabolites that are beneficial to the host animal.

In certain embodiments, the beneficial microbiome metagenomic function comprises pathways and metabolites responsible for recovering metabolic energy from otherwise undigested or unutilized components of the animals' diets. In some variations, the undigested or unutilized components of the animals' diet comprises fiber, non-starch polysaccharides, digestion-resistant carbohydrates, hemicellulosic species, pectins, fiber-bound protein, fiber-bound micronutrients, and chelated minerals or metals. In certain embodiments, the beneficial microbiome metagenomic function is the “C₃ Pathway” associated with the production of gluconeogenic metabolites, which can be absorbed by the animal and recovered as metabolic energy. In particular embodiments, the C₃ Pathway is defined by the total abundance of genes in the metagenome annotated by the E.C. numbers selected from the list of E.C. numbers consisting of 1.1.1.27, 1.2.1.87, 1.3.1.95, 2.8.3.1, and 4.2.1.28

In particular embodiments, the beneficial microbiome metagenomic function is the “C₄ Pathway” associated with the production of butyrate and other short-chain fatty acids that provide direct nourishment for epithelial cells and promote a healthy inflammatory response by the animal. In particular embodiments, the C₄ Pathway is defined by the total abundance of genes in the metagenome annotated by the E.C. numbers selected from the list of E.C. numbers consisting of 1.1.1.35, 1.1.1.36, 1.1.1.61, 1.2.1.76, 1.3.8.1, 2.3.1.247, 2.8.3.1;2.8.3.8, 2.8.3.18, 2.8.3.9, 3.1.2.4, 4.2.1.150, 4.2.1.55, and 4.3.1.14.

The Se-VitE preparation, nutritional composition or animal feed premix may be provided to the animal on any appropriate schedule. In some embodiments, the animal is provided the Se-VitE preparation, nutritional composition or animal feed premix on a daily basis, on a weekly basis, on a monthly basis, on an every other day basis, for at least three days out of every week, or for at least seven days out of every month.

In some embodiments, the nutritional composition, the Se-VitE preparation or the animal feed premix is administered to the animal multiple times in a day. For examples, in some embodiments, the nutritional composition, the Se-VitE preparation or the animal feed premix is administered to the animal at least 1, 2, 3, 4, 5, 6, 7, 8, 9, or 10 times a day.

The present invention will be further illustrated by the following examples.

Sampling and Detecting

A. Sampling and Detecting Gastrointestinal Microbes

In certain embodiments, the methods described herein include detecting or quantifying one or more microbial (e.g., bacterial) genuses in the gastrointestinal microbiota of an animal. In certain embodiments, the microbial (e.g., bacterial) genus is detected or quantified in a gastrointestinal microbiota sample from an animal. Gastrointestinal microbiota samples can be obtained from an animal in any standard form which reflects the microbial contents of the gastrointestinal tract of the animal. Gastrointestinal microbiota samples include gastrointestinal tissue samples obtained e.g., by endoscopic biopsy. Gastrointestinal tissues include, e.g., oral tissue, esophagus, stomach, intestine, ileum, cecum, colon or rectum. Samples also feces, saliva, and gastrointestinal ascites. Methods of obtaining gastrointestinal microbiota samples are standard and known to the skilled artisan.

In some embodiments, the sample is a single sample from a single animal. In some embodiments, the sample is a combination of multiple samples from a single animal. In some embodiments, microbes (e.g., bacteria (e.g., total bacteria)) are purified from the sample prior to analysis. In some embodiments, microbes (e.g., bacteria) from a single sample are purified. In some embodiments, microbes (e.g., bacteria) from multiple samples from a single animal are purified and subsequently combined prior to analysis.

In some embodiments, total DNA or total RNA is isolated from the sample. Genomic DNA can be extracted from samples using standard methods known to the skilled artisan and including commercially available kits, such as the Mo Bio Powersoil®-htp 96 Well Soil DNA Isolation Kit (Mo Bio Laboratories, Carlsbad, CA), the Mo Bio Powersoil® DNA Isolation Kit (Mo Bio Laboratories, Carlsbad, CA), or the QIAamp DNA Stool Mini Kit (QIAGEN, Valencia, CA) according to the manufacturers instructions. RNA can be extracted from samples using standard assays known to the skilled artisan including commercially available kits, such as the RNeasy PowerMicrobiome Kit (QIAGEN, Valencia, CA) and RiboPure Microbial RNA Purification Kit (Life Technologies, Carlsbad, CA). Another method for isolation of microbial (e.g., bacterial) RNA may involve enrichment of mRNA in purified samples of microbial RNA through removal of tRNA. Alternatively, RNA may be converted to cDNA, which can be used to generate sequencing libraries using standard methods such as the Nextera XT Sample Preparation Kit (Illumine, San Diego, CA).

Identification and determination of the relative abundance of a microbial (bacterial) genus in a sample may be determined by standard molecular biology methods known to the skilled artisan, including e.g., genetic analysis (e.g. DNA sequencing (e.g., full genome sequencing, whole genome shotgun sequencing (WSG)), RNA sequencing, PCR, quantitative PCR (qPCR)), serology and antigen analysis, microscopy, metabolite identification, gram staining, flow cytometry, immunological techniques, and culture based methods such as counting colony forming units.

In some embodiments, identification and relative abundance of a microbial (e.g., bacterial) genus is determined by whole genome shot gun sequencing (WGS), wherein extracted DNA is fragmented into pieces of various lengths (from 300 to about 40,000 nucleotides) and directly sequenced without amplification. Sequence data can be generated using any sequencing technology including for example, but not limited to Sanger, Illumine, 454 Life Sciences, Ion Torrent, ABI, Pacific Biosciences, and/or Oxford Nanopore.

Sequencing libraries for microbial whole-genome sequencing (WGS) may be prepared from microbial (e.g., bacterial) genomic DNA. For genomic DNA that has been isolated from an animal sample, the DNA may optionally be enriched for microbial DNA using commercially available kits, for example, the NEBNext Microbiome DNA Enrichment Kit (New England Biolabs, Ipswich, MA) or other enrichment kit. Sequencing libraries may be prepared from the genomic DNA using commercially available kits as well, such as the Nextera Mate-Pair Sample Preparation Kit, TruSeq DNA PCR-Free or TruSeq Nano DNA, or the Nextera XT Sample Preparation Kit (Illumina, San Diego, CA) according to the manufacturer's instructions.

Alternatively, libraries can be prepared using other kits compatible with the Illumina sequencing platform, such as the NEBNext DNA Library Construction Kit (New England Biolabs, Ipswich, MA). Libraries may then be sequenced using standard sequencing technology including, but not limited to, a MiSeq, HiSeq or NextSeq sequencer (Illumina, San Diego, CA).

Alternatively, a whole genome shotgun fragment library prepared using standard methods in the art may be used. For example, the shotgun fragment library could be constructed using the GS FLX Titanium Rapid Library Preparation Kit (454 Life Sciences, Branford, CT), amplified using a GS FLX Titanium emPCR Kit (454 Life Sciences, Branford, CT), and sequenced following standard 454 pyrosequencing protocols on a 454 sequencer (454 Life Sciences, Branford, CT).

Nucleic acid sequences can be analyzed to define taxonomic assignments using sequence similarity and phylogenetic placement methods or a combination of the two strategies. A similar approach can be used to annotate protein names, protein function, transcription factor names, and any other classification schema for nucleic acid sequences. Sequence similarity based methods include BLAST, BLASTx, tBLASTn, tBLASTx, RDP-classifier, DNAclust, RapSearch2, DIAMOND, USEARCH, and various implementations of these algorithms such as QIIME or Mothur. These methods map a sequence read to a reference database and select the best match. Common databases include KEGG, MetaCyc, NCBI non-redundant database, Greengenes, RDP, and Silva for taxonomic assignments. For functional assignments, reads are mapped to various functional databases such as COG, KEGG, BioCyc, MetaCyc, and the Carbohydrate-Active Enzymes (CAZy) database. Microbial clades are assigned using software including MetaPhlAn.

In some embodiments, the microbial constituents are identified by characterizing the DNA sequence of microbial 16S small subunit ribosomal RNA gene (16S rRNA gene). 16S rRNA gene is approximately 1,500 nucleotides in length, and in general is highly conserved across organisms, but contain specific variable and hypervariable regions (V1-V9) that harbor sufficient nucleotide diversity to differentiate genus- and strain-level taxa of most organisms. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature.

Composition of a microbial community can be deduced by sequencing full 16S rRNA gene, or at least one of the VI, V2, V3, V4, V5, V6, V7, V8, and V9 regions of this gene or by sequencing of any combination of variable regions from this gene (e.g. VI-3 or V3-5). In one embodiment, the VI, V2, and V3 regions are used to characterize a microbiota. In another embodiment, the V3, V4, and V5 regions are used to characterize a microbiota. In another embodiment, the V4 region is used to characterize a microbiota.

Sequences that are at least 97% identical to each other are grouped into Operational Taxonomic Units (OTUs). OTUs that contain sequences with 97% similarity correspond to approximately genus level taxa. At least one representative sequence from each OTU is chosen, and is used to obtain a taxonomic assignment for an OTU by comparison to a reference database of highly curated 16S rRNA gene sequences (such as Greengenes or SILVA databases). Relationship between OTUs in a microbial community could be deduces by constructing a phylogenetic tree from representative sequences from each OTU. Using known techniques, in order to determine the full 16S sequence or the sequence of any variable region of the 16S sequence, genomic DNA is extracted from a microbial sample, the 16S rRNA (full region or specific variable regions) amplified using polymerase chain reaction (PCR), the PCR products are cleaned, and nucleotide sequences delineated to determine the genetic composition of 16S rRNA gene or a variable region of the gene. If full 16S sequencing is performed, the sequencing method used may be, but is not limited to, Sanger sequencing. If one or more variable regions is used, such as the V4 region, the sequencing can be, but is not limited to being performed using the Sanger method or using a next-generation sequencing method, such as an Illumine method. Primers designed to anneal to conserved regions of 16S rRNA genes (e.g., the 515F and 805R primers for amplification of the V4 region) could contain unique barcode sequences to allow characterizing multiple microbial communities simultaneously.

In addition to the 16S rRNA gene, a selected set of genes that are known to be marker genes for a given genus or taxonomic group is analyzed to assess the composition of a microbial community. These genes are alternatively assayed using a PCR-based screening strategy. For example, various strains of pathogenic Escherichia coli are distinguished using genes that encode heat-labile (LTI, LTIIa, and LTIIb) and heat-stable (STI and STII) toxins, verotoxin types 1, 2, and 2e (VT1, VT2, and VT2e, respectively), cytotoxic necrotizing factors (CNF1 and CNF2), attaching and effacing mechanisms (eaeA), enteroaggregative mechanisms (Eagg), and enteroinvasive mechanisms (Einv). The optimal genes to utilize to determine the taxonomic composition of a microbial community by use of marker genes are familiar to one with ordinary skill in the art of sequence based taxonomic identification.

In some embodiments, the identity of the microbial composition is characterized by identifying nucleotide markers or genes, in particular highly conserved genes (e.g., “house-keeping” genes), or a combination thereof. Using defined methods, DNA extracted from a microbial sample will have specific genomic regions amplified using PCR and sequenced to determine the nucleotide sequence of the amplified products.

B. Sampling and Detecting Gastrointestinal Metabolites

Gastrointestinal samples can be obtained from an animal in any standard form which reflects the metabolic contents of the gastrointestinal tract of the animal. Gastrointestinal samples include gastrointestinal tissue samples obtained e.g., by endoscopic biopsy. Gastrointestinal tissues include, e.g., oral tissue, esophagus, stomach, intestine, ileum, cecum, colon or rectum. Samples also feces, saliva, and gastrointestinal ascites. Methods of obtaining gastrointestinal samples are standard and known to the skilled artisan.

In some embodiments, the sample is a single sample from a single animal. In some embodiments, the sample is a combination of multiple samples from a single animal. In some embodiments, metabolites are purified from the sample prior to analysis. In some embodiments, metabolites from a single sample are purified. In some embodiments, metabolites from multiple samples from a single animal are purified and subsequently combined prior to analysis.

The metabolites that are present in gastrointestinal samples collected from animals or in fresh or spent culture media may be determined using methods described herein and known to the skilled artisan. Such methods include for example chromatography (e.g., gas (GC) or liquid chromatography (LC)) combined with mass spectrometry or NMR (e.g., 1H-NMR). The measurements may be validated by running metabolite standards through the same analytical systems.

In the case of gas chromatography-mass spectrometry (GC-MS) or liquid-chromatography-mass spectrometry (LC-MS) analysis, polar metabolites and fatty acids could be extracted using monophasic or biphasic systems of organic solvents and an aqueous sample and derivatized. An exemplary protocol for derivatization of polar metabolites involves formation of methoxime-tBDMS derivatives through incubation of the metabolites with 2% methoxylamine hydrochloride in pyridine followed by addition of N-tert-butyldimethylsilyl-N-methyltrifluoroacetamide (MTBSTFA) with 1% tert-butyldimethylchlorosilane (t-BDMCS). Non-polar fractions, including triacylglycerides and phospholipids, may be saponified to free fatty acids and esterified to form fatty acid methyl esters, for example, either by incubation with 2% H₂SO₄ in methanol or by using Methyl-8 reagent (Thermo Scientific). Derivatized samples may then be analyzed by GC-MS using standard LC-MS methods, for example, a DB-35MS column (30 m×0.25 mm i.d.×0.25 pin, Agilent J&W Scientific) installed on a gas chromatograph (GC) interfaced with a mass spectrometer (MS). Mass isotopomer distributions may be determined by integrating metabolite ion fragments and corrected for natural abundance using standard algorithms. In the case of liquid chromatography-mass spectrometry (LC-MS), polar metabolites may be analyzed using a standard benchtop LC-MS/MS equipped with a column, such as a SeQuant ZIC-Philic polymeric column (2.1×150 mm; EMD Millipore). Exemplary mobile phases used for separation could include buffers and organic solvents adjusted to a specific pH value.

In combination or in the alternative, extracted samples may be analyzed by ¹H-nuclear magnetic resonance (¹H-NMR). Samples may be combined with isotopically enriched solvents such as D₂O, optionally in the presence of a buffered solution (e.g., Na₂HPO₄, NaH₂PO₄ in D₂O, pH 7.4). Samples may also be supplemented with a reference standard for calibration and chemical shift determination (e.g., 5 mM 2,2-dimethyl-2-silapentane-5-sulfonate sodium salt (DSS-d₆, Isotec, USA)). Prior to analysis, the solution may be filtered or centrifuged to remove any sediment or precipitates, and then transferred to a suitable NMR tube or vessel for analysis (e.g., a 5 mm NMR tube). ¹H-NMR spectra may be acquired on a standard NMR spectrometer, such as an Avance II+500 Bruker spectrometer (500 MHz) (Bruker, DE), equipped with a 5 mm QXI-Z C/N/P probe-head) and analyzed with spectra integration software (such as Chenomx NMR Suite 7.1; Chenomx Inc., Edmonton, AB). Alternatively, ¹H-NMR may be performed following other published protocols known in the art (see e.g., Chassaing et al., Lack of soluble fiber drives diet-induced adiposity in mice, Am J Physiol Gastrointest Liver Physiol, 2015; Bai et al., Comparison of Storage Conditions for Human Vaginal Microbiome Studies, PLoS ONE, 2012:e36934).

C. Microbial Gene Transcription

Microbial (e.g., bacterial) proteins described herein can be detected and quantified using standard molecular biology techniques. For example, the level of expression on a microbial protein can be detected in a gastrointestinal sample of an animal by RNA (e.g. RNA sequencing, shotgun sequencing, quantitative PCR) or protein expression (e.g. ELISA, western blot, other immunological techniques).

### Example

Cecum samples (poultry) were been collected from euthanized broilers at day 35 (description of VT diet). Eight samples were collected from control animals, eight samples were collected from animals with diet enriched in vitamin E and selenium. Cecum slurries were flash frozen in dry ice after collection and stored at −80° C.

After DNA extraction, 16S Sv4 ribosomal RNA sequencing was performed at Diversigen (Houston, TX) on an Illumine MiSeq sequencer. Raw sequences were processed using the Diversigen 16S rRNA pipeline build around operational taxonomic units (OUT) identification on the SILVA database.

Compositional data (relative abundances) were analyzed using features ranking Songbird procedure which was developed to alleviate microbial load variations amongst different samples and focuses on log-fold change in relative abundance with respect to co-variates or conditions (Morton et al., 2019, Establishing microbial composition measurement standards with reference frames. Nature Communications https://doi.org/10.1038/s41467-019-10656-5). Visualization of feature ranking was performed using Qurro (Fedarko et al., 2020, Visualizing 'omics feature rankings and log-ratios using Qurro. NAR Genomics and Bioinformatics 2:1-7.)

Ninety features could be computed at the genus level and ranking with regards to their association to the vitamin E and Selenium treatment (Table 1). Their associated feature value were calculated through Songbird and visualized through Qurro in FIG. 1 which shows rank plot with features positively or negatively associated to treatment with vitE and Se. Fold changes were measured wrt control. In red, Lactobacillus, Coprococcus and Faecalibacterium features. In blue, Escherichia coli and Bacteroides features.

Of interest as the highest negatively associated with vitamin E and Selenium was Escherichia coli. Another interesting genus negatively associated with vit E and Sel was Bacteroides. On the positive association side, one Coprococcus, two Lactobacillus were significantly observed. Faecalibacterium positively associated feature value was weaker. Both Coprococcus and Faecalibacterium are remarquable butyrate producers that plain an important role in healthy gut host biology. Lactobacilli strains have many interesting metabolic behavior, from lactate production to bacteriocin or bile acid enzyme degradation.

TABLE 1
Feature differentials computed through Songbird

| featureid | Intercept | treatment | |
|---|---|---|---|
| GBKCeco5 | 0.696364 | −0.54435 | |
| Unc20158 | 2.01536 | −0.31228 | |
| Unc012os | −0.78738 | 1.029906 | |
| Unc15463 | 0.629839 | −0.54358 | |
| UncG3786 | 2.997475 | −2.09441 | Escherichia Shigella |
| UncCI398 | −0.94887 | −0.26695 | |
| UncTer61 | −0.26743 | 1.050717 | |
| Unc054vi | 3.941097 | −0.54109 | Bacteroides |
| Unc03j0p | 2.670065 | −0.96355 | |
| Unc39231 | −1.30831 | 0.136107 | |
| Unc00wmn | 1.070422 | −1.06966 | |
| Unc70424 | −0.49245 | −1.16119 | |
| G7WHuG28 | −0.1172 | −0.56748 | |
| Unc0330q | −0.21439 | 0.231119 | |
| Unc02dvr | 0.618246 | 0.200592 | |
| Unc022wn | −0.83284 | 0.000887 | |
| Unc004yi | −0.56781 | −0.31882 | Lactobacillus |
| Unc37391 | 2.194487 | −0.31683 | |
| Unc02vwv | −1.33916 | −0.29548 | |
| Unc65259 | 1.386566 | 0.360176 | |
| FNWIc111 | −0.58924 | 0.079591 | |
| Unc77247 | −0.5466 | 0.133385 | |
| Unc00xgb | −1.97743 | 0.662695 | |
| Unc010oz | 0.267255 | 0.348092 | |
| Unc76755 | −0.2304 | 0.85814 | |
| Unc0155b | −1.69784 | 0.295096 | |
| Unc02741 | −1.42776 | 0.463787 | |
| Unc00zah | −0.56453 | −0.47509 | |
| Unc0317l | 0.562918 | −1.37741 | |
| Unc02308 | −0.86272 | −0.45101 | |
| IC0Bact7 | −1.02507 | −0.65875 | |
| Unc015e7 | −1.23647 | −0.49212 | |
| Unc01cjl | −1.45892 | −0.18928 | |
| Unc03s2x | −0.21305 | 0.153553 | |
| A6BStoq4 | 0.625966 | −0.8095 | |
| Unc04y8f | −1.14632 | 0.227219 | |
| Unc05ewv | 0.199569 | −0.34816 | |
| Unc00nqc | 0.433666 | 0.186544 | |
| Unc02g4i | −1.29018 | 1.12013 | |
| GJFBac89 | −0.94386 | 0.271249 | |
| Unc86155 | 2.824916 | −0.20499 | |
| Unc056wt | 0.362214 | 0.650852 | Lactobacillus |
| Unc68159 | −0.19614 | 0.183845 | |
| Unc38154 | −0.67041 | −0.34416 | |
| UncFi187 | 0.408508 | 0.150596 | |
| Unc87852 | −0.70254 | 0.740245 | |
| Unc00z31 | −1.39484 | 1.884652 | |
| Unc79056 | −0.32902 | −0.28413 | |
| Unc01bsz | −0.38162 | 0.66992 | |
| Unc80743 | −1.26689 | 0.16928 | |
| LcbSal24 | 0.01958 | 0.591653 | Lactobacillus |
| Unc75688 | 0.870781 | −0.19912 | |
| Unc01de7 | −1.60498 | 0.251674 | |
| Unc01bfz | −1.02085 | 0.36048 | |
| Unc004oc | 0.265107 | −0.5244 | |
| UncO9245 | 0.359883 | 0.622855 | Coprococcus |
| Unc89438 | −1.28439 | 0.170982 | |
| UncB2938 | 0.601857 | −0.81911 | |
| Unc02fcj | 0.872415 | 0.371476 | |
| Unc99454 | 0.989648 | 0.088165 | |
| Unc053cd | −0.53192 | −0.18892 | |
| UncO5247 | 0.170824 | −0.97702 | |
| FNWFB201 | −0.34628 | −0.39564 | |
| Unc03jsh | 1.609411 | −0.66597 | |
| Unc77180 | −1.40165 | 1.091515 | |
| Unc05eti | 0.486638 | 0.442273 | |
| Unc04w4q | −0.40181 | 0.016541 | |
| Unc77683 | 1.654256 | −0.80293 | |
| Unc02g9a | −0.91092 | 0.46885 | |
| Unc37284 | 2.465683 | −1.10018 | |
| H9MBac20 | −1.64623 | 0.952696 | |
| Unc01vbr | −1.58643 | 0.075655 | |
| Unc30909 | −0.68748 | 0.239063 | |
| Unc04ez7 | −1.00586 | 0.539093 | |
| Unc014fz | 0.762497 | 0.63362 | |
| Unc02n51 | 1.929328 | −1.60486 | |
| Unc22440 | −1.16037 | 0.636981 | |
| Unc0162b | −1.44727 | 0.581914 | |
| GJFBa110 | −0.57391 | 0.245879 | |
| Unc015ac | 0.51882 | 0.135998 | |
| Unc99710 | 0.87844 | 0.054961 | |
| Unc56686 | 1.095572 | −1.20112 | |
| UncB6702 | 1.040361 | −0.41113 | |
| Unc013u3 | 3.874128 | 0.131421 | |
| Unc01heh | −1.43708 | −0.0685 | |
| Unc00ru4 | −0.68678 | 0.438105 | |
| Unc000fi | −1.70848 | 0.734129 | |
| Unc02qbx | 0.226213 | 0.5031 | |
| Unc012jr | −1.00219 | 0.855251 | |
| Unc91505 | 2.876184 | 0.096478 | Faecalibacterium |

1. A method of increasing gut relative abundance of beneficial gut microorganisms, the method comprising the step of administering a nutritional composition comprising a base nutritional composition and a Se-VitE preparation to the animal, wherein the level of one or more microbial genuses in the gastrointestinal tract of the animal is higher relative to the level of the microbial genuses in the gastrointestinal tract of an animal administered a nutritional composition lacking the Se-VitE preparation.

2. A method of promoting the growth of one or more beneficial microbial genuses in the gastrointestinal tract of an animal, the method comprising the step of administering a nutritional composition comprising a base nutritional composition and a Se-VitE preparation to the animal, wherein the level of one or more microbial genuses in the gastrointestinal tract of the animal is higher relative to the level of the microbial genuses in the gastrointestinal tract of an animal administered a nutritional composition lacking the Se-VitE preparation.

3. The method of claim 1, wherein the microbial genus is a bacterial genus.

4. The method of claim 1, wherein the microbial genus is selected from the group consisting of: Lactobacilli, Coprococcus, Faecalibacterium.

5. A method for selectively promoting or inhibiting the production of one or more gastrointestinal metabolites in an animal, the method comprising the step of administering a nutritional composition and a Se-VitE preparation to said animal, wherein the level of the at least one metabolites is associated with animal performance, health and welfare and is higher or lower in a gastrointestinal sample from said animal compared to a gastrointestinal sample from a comparable control animal that has been administered a comparable nutritional composition comprising said nutritional composition and lacking said Se-VitE preparation.

6. A method for improving nitrogen utilization in an animal, the method comprising: administering a nutritional composition comprising a base nutritional composition and a Se-VitE preparation to said animal, wherein the level of a plurality of metabolites associated with enhanced nitrogen utilization is higher in a gastrointestinal sample from said animal compared to a gastrointestinal sample from a comparable control animal that has been administered a comparable nutritional composition comprising said base nutritional composition and lacking said Se-VitE preparation.

7. A method of improving carbon utilization in an animal, the method comprising: administering a nutritional composition comprising a base nutritional composition and a Se-VitE preparation to said animal, wherein the level of a plurality of metabolites associated with improved carbon utilization in a gastrointestinal sample from said animal is higher compared to a comparable control animal that has been administered a comparable nutritional composition comprising said base nutritional composition and lacking said Se-VitE preparation.

8. A method of decreasing gut relative abundance of non-beneficial gut microorganisms, the method comprising the step of administering a nutritional composition comprising a base nutritional composition and a Se-VitE preparation to the animal, wherein the level of one or more non-microbial genuses in the gastrointestinal tract of the animal is lower relative to the level of the microbial genuses in the gastrointestinal tract of an animal administered a nutritional composition lacking the Se-VitE preparation.

9. A method of suppressing the growth of one or more non-beneficial microbial genuses in the gastrointestinal tract of an animal, the method comprising the step of administering a nutritional composition comprising a base nutritional composition and a Se-VitE preparation to the animal, wherein the level of one or more non beneficial microbial genuses in the gastrointestinal tract of the animal is lower relative to the level of the microbial genuses in the gastrointestinal tract of an animal administered a nutritional composition lacking the Se-VitE preparation.

10. The method of claim 8, wherein the microbial genus is a bacterial genus.

11. The method of claim 8, wherein the microbial genus is selected from the group consisting of: Escherichia coli, Bacteroides and Prevotella.

12. The method of claim 1, wherein the nutritional composition comprising the Se-VitE preparation is administered to the animal for at least 1, 7, 10, 14, 30, 45, 60, 90, or 120 days.

13. The method of claim 1, wherein the nutritional composition comprising the Se-VitE preparation is administered to the animal at least once, twice, three, four, or five times a day.

14. The Se-VitE preparation of claim 1, wherein vitamin E is selected from the group consisting of d-α-tocopherol, dl-α-tocopherol, d-α-tocopherol acetate, dl-α-tocopherol acetate, d-α-tocopherol succinate and/or dl-α-tocopherol succinate.

15. The preparation of claim 14, wherein selenium is in an inorganic form such as sodium selenite (Na₂SeO₃) and sodium selenite (Na₂SeO₄), or in an organic form such as selenomethionine.

16. The preparation of claim 14, wherein the composition, and/or vitamin E and or selenium is formulated as a liquid formulation or a solid formulation.

17. An animal feed, comprising the preparation of claim 14, wherein
a. vitamin E is contained in an amount of from 50 mg to 500 mg, preferably from 80 mg to 450 mg, more preferably from 100 mg to 400 mg, more preferably from 120 mg to 350 mg, even more preferably from 150 mg to 300 mg or most preferably from 180 mg to 300 mg such as 180, 200, 210, 220, 230, 240, 250, 260, 270, 280, 290 and 300 mg, per 1 Kg of the animal feed
b. selenium is contained in an amount of from 0.5 mg to 5 mg, preferably from 0.6 mg to 4.0 mg, more preferably from 0.7 mg to 3.0 mg, more preferably from 0.75 mg to 2.5 mg, even more preferably from 0.8 mg to 2.0 mg or most preferably from 0.85 mg to 1.5 mg such as 0.9, 0.95, 1.0, 1.05, 1.1, 1.15, 1.2, 1.25, 1.3, 1.35, 1.4, 1.45 and 1.5 mg, per 1 Kg of the animal feed.
